(19) United States
(12) Patent Application Publication
(10) Pub. No.: US 2009/0022788 A1
(43) Pub. Date: Jan. 22, 2009

(54) DIETARY SUPPLEMENT FOR HEALING OR REGRESSING SYMPTOMS OF GASTROESOPHAGEAL REFLUX DISEASE, GASTRITIS AND ULCERS

(76) Inventor: Ricardo de souza PEREIRA, Sao Paulo (BR)

Correspondence Address: CANTOR COLBURN, LLP, 20 Church Street, 22nd Floor, Hartford, CT, 06103, US

(21) Appl. No.: 12/241,759

(22) Filed: Sep. 30, 2008

Related U.S. Application Data
(63) Continuation of application No. PCT/BR2007/000012, filed on Jan. 19, 2007.

(30) Foreign Application Priority Data
Mar. 30, 2006 (BR) PI 0601834-3

(51) Int. Cl. A61K 9/48; A61K 31/714; A61P 1/04

(52) U.S. Cl. 424/456; 514/52

(57) ABSTRACT

The current invention presents a dietary supplement obtained from a mixture of melatonin, vitamins and aminoacids for healing or regressing symptoms of gastroesophageal reflux disease (such as heartburn, regurgitation, dysphagia, coughing, hoarseness, chest pain and odynophagia), gastritis and several types of ulcerations.

This application is a continuation of PCT Application No. PCT/BR2007/000012 filed on 19 Jan. 2007, the contents of said application are herein incorporated by reference in their entirety.

The current invention presents a DIETARY SUPPLEMENT FOR HEALING OR REGRESSING SYMPTOMS OF GASTROESOPHAGEAL REFLUX DISEASE, GASTRITIS AND ULCERS, which can be used for healing gastroesophagel reflux disease (GERD), gastritis and various types of ulcerations.

### BACKGROUND OF THE INVENTION

Gastroesophageal reflux disease (GERD) is the reflux of gastric contents into the esophagus and/or adjacent organs, with or without tissue damage [Moraes-Filho, J., et al., Am. J. Gastroenterol., 97: 241-8 (2002)]. GERD is a common condition, with an estimated 44% of the adult population in USA experiencing its symptoms monthly [Fass, R., Am. J. Gastroenterol., 98 (Suppl.): S2-S7 (2003)]. In Brazil, there is no national study done with adequate and specific statistic analysis [Nader, F., et al., Arq. Gastroenterol., 40: 31-34 (2003)].

The most prominent symptom of GERD is heartburn, with or without regurgitation of gastric contents into the mouth. Irritation of the lining of the esophagus by gastric acid secretions (stomach acid) causes Barrett's esophagus. Optimal medical management of Barrett's esophagus is uncertain [Hillman, L. C., et al., Med. J. Aust., 180: 387-391 (2004)]. Asymptomatic Barrett's esophagus presents no indications to initiate treatment, which in symptomatic patients is carried out in the normal fashion [Koop, H., Chirurg, 76: 353-358 (2005)]. Barrett's esophagus is a premalignant condition, with dysplasia usually preceding the development of adenocarcinoma. Although there is evidence that anti-reflux surgery results in regression of dysplasia and possibly prevents the development of high-grade dysplasia and adenocarcinoma [Hofstetter, W. L., et al., Ann. Surg., 234: 532-539 (2001); Koop, H., Endoscopy, 34: 97-103 (2002)], persistent reflux may continue to produce proliferative activity and more dysplasia [Chen, L. Q., et al., Ann. Surg., 234: 172-180 (2001)].

Recent data suggest that nonsteroidal anti-inflammatory drugs (NSAIDs), such as aspirin, may prevent the progression of Barrett's esophagus to adenocarcinoma. However, use of aspirin is associated with numerous potential complications, including gastrointestinal bleeding and hemorrhagic strokes [Hur, C., et al., J. Natl. Cancer Inst., 96: 316-325 (2004)]. Gastrointestinal bleeding remains a substantial cause of morbidity and mortality (Pisegna, J. R., Pharmacotherapy, 10 Pt2: 81S-86S (2003)].

Acid-suppressant drugs predominate in the treatment of GERD. Proton pump inhibitors (PPI) are the first-line choice in both reflux esophagitis and nonerosive reflux disease (NERD). H(2)-blockers play a minor role and should not be used in erosive esophagitis. Other drugs, such as mucosa-protective compounds, prokinetics, and antacids do not play a role, either alone or in combination with acid suppressants. Proton pump inhibitors, such as lanzoprazole and omeprazole, are also used in maintenance therapy [Koop, H., Chirurg, 76: 353-358 (2005); Der, G., Gastroenterol. Nurs., 26: 182-190 (2003)].

Omeprazole (U.S. Pat. No. 4,255,431; Eur. Patent Appl. 5,129), rabeprazole (U.S. Pat. No. 5,045,552; Eur. Patent Appl. 268,956), pantoprazole (U.S. Pat. No. 4,758,579; Eur. Patent Appl. 166,287) and lanzoprazole (U.S. Pat. No. 4,628,098; Eur. Patent Appl. 174,726) have potentially serious adverse side effects. They abolish acid production so completely that serum gastrin levels rise. In rodents enterochromaffin-like cell tumors and carcinoid tumors have developed. It is not known whether these drugs are carcinogenic in humans by a similar mechanism [Viste, A., et al., Gastric Cancer, 7: 31-35 (2004)]. Moreover, bacterial overgrowth may develop in the stomach in the absence of acid. Bacterial metabolism of dietary nitrites may then lead to the production of N-nitroso compounds that are carcinogenic [Viste, A., et al., Gastric Cancer, 7: 31-35 (2004); Vermeer, I. T., et al., Gastroenterology, 121: 517-25 (2001)]. This risk is not limited to chronic omeprazole treatment; it can theoretically occur with any effective long-term antacid regimen. Moreover, omeprazole appears to affect cytochrome P450. Although initial studies suggested an inhibitory effect, more recent studies indicate that omeprazole may induce the cytochrome P450 1A subfamily that is associated with activation of certain chemical procarcinogens, such as polycyclic aromatic hydrocarbons [Kalant H., Roschlau W. H. E. (1998): Principles of Medical Pharmacology, 6^{th} edition: Oxford University Press, New York, pp. 558].

### AIM OF THE INVENTION

In order to try to promote the regression of these gastroenterological disorders, a formulation with vitamins and aminoacids, which have no side effects was developed. This formulation is based on information existing in scientific literature [Waterland, R. A. and Jirtle, R. L., Nutrition, 20: 63-68 (2004); Fetrow, C. W. and Avila, J. R., Ann. Pharmacother., 35: 1414-25 (2001)] and accumulated by my research group [Pereira, R. S., FEBS Lett., 475: 43-46 (2000); Pereira, R. S., Biochem. Pharmacol., 62: 975-983 (2001)]. The compounds of this formula can be used by any person (included those with asymptomatic Barrett's esophagus).

### DETAILED DESCRIPTION

The dietary supplement of the current invention, has its origin in natural medicine and it is based on reposal of melatonin, vitamins and aminoacids which are lost during a strong emotional stress: kidnapping, assault, raping, accidents (car, bus, train, airplane, etc.), death of a loved person, divorce, fighting in wars, lost of limbs, use of alcohol and/or drugs, or other origin that, until the present moment, can be unknown and can change the personality of a person each passing day, inducing the appearing of gastroesophageal reflux disease, ulcer or gastritis.

Vitamin B12 can be obtained from fermentation process using Pseudomonas denitrificans (U.S. Pat. No. 3,018,225) and from cultures of Streptomyces griseus (U.S. Pat. No. 2,563,794), Betaine and Folic Acid are obtained by the general method described in U.S. Pat. No. 2,800,502 and U.S. Pat. No. 2,956,057, respectively. These and all of them are available commercially, which are hereby incorporated by reference.

### Formulation:

3-Hydroxy-2-methyl-5-([phosphonooxy]methyl)-4-pyridinecarboxaldehyde (Vitamin B6) . . . from 1 μg to 10.000 mg
L-α-amino-3-indolepropionic acid or 3 β-indolylalanine (Tryptophan) . . . from 1 μg to 10.000 mg
(Carboxymethyl)trimethylammonium (Betaine) . . . from 1 μg to 10.000 mg
Pteroyl-L-glutamic acid (Folic Acid or Vitamin M) . . . from 1 μg to 10.000 mg
α(5,6-dymethylbenzimidazolyl) cyanocobamide (Vitamin B12) . . . from 1 μg to 10.000 mg
N-Acetyl-5-methoxytryptamine (Melatonin) . . . from 1 μg to 10.000 mg
L-2-Amino-4-(methylthio)butanoic acid (Methionine) . . . from 1 μg to 10.000 mg

Such biochemicals are weighed and encapsulated in gelatin capsule by a trained pharmacist using an automatic or manual encapsulator.

The patient should take 1 or 2 capsules a day (or according medical recommendation), at bedtime (after a meal) with water or milk, from 01 to 200 days or while the patient is feeling the symptoms. This formulation can be used in other pharmaceutical preparations: pills, tablets, syrups, solutions, injectables, or other similars that can be invented in the future.

This formulation was tested in 351 patients who had GERD, ulcers or heartburn. Once finished the treatment, the patients did not feel need to continue taking it. According their reports, the pain, heartburn, dysphagia, regurgitation and other symptoms disappeared. A total healing was observed in 99% of the cases and in the remaining case, a significant regressing of the symptoms: an esophagitis grade 4 regressed to a grade 1, after nine months of treatment [Pereira, R. S., J. Pineal Res., 41: 195-200 (2006)].

For example, a 35-year-old white male patient with chronic GERD had, initially, regurgitation and heartburn. He had these symptoms for more than two years and thereafter he treated himself with baking soda and antacids. Thereafter, a gastroenterologist prescribed omeprazole. Neither treatment produced even partial relief of his symptoms. Subsequently, he started to lose weight because he could no longer eat properly due to the acute pain impaired swallowing and massive hematemesis. Even when he tried to drink an apple blended with water, he vomited blood. Endoscopic examinations showed that he had an ulcer of 6 cm in the esophagus. As a consequence, he lost 40 kilograms in 6 months. After he consulted 5 gastroenterologists, he brought to me his endoscopy record and medical report. I immediately prescribed the following formulation described above. Two hours after he took the first gelatin capsule, his pain regressed. He told me that he was hungry and he bought a roasted chicken which he ate in its entirely, without feeling pain. After 32 days of treatment, he had recovered 30 kilos. In photos taken in October and November of 2003, the color of his hair was yellow. The patient reported that his hair had changed color from black to yellow after the ulcer appeared (he did not dye his hair). This is consistent with the observations in rats where malnutrition changed hair color to yellow [Waterland, R. A. and Jirtle, R. L., Nutrition, 20: 63-68 (2004)]. After treatment, the patient recovered the natural color of hair [Pereira, R. S., J. Pineal Res., 40: 355-356 (2006); Pereira, R. S., J. Pineal Res., 41: 195-200 (2006)].

The patient stopped taking omeprazole to avoid malabsorption of vitamin B12 (present in the formula), since it has previously been reported that omeprazole (and other PPIs) and histamine (2)-receptor antagonists are associated with vitamin B12 deficiency; the concentration of this vitamin may be decreased when gastric acid is markedly suppressed for prolonged periods [Valuck, R. J. and Ruscin, J M., J. Clin. Epidemiol., 57: 422-428 (2004)].

At the end of treatment, the patient had taken 280 capsules over nine months. The patient underwent endoscopy and biopsy again in order to evaluate the healing of active ulcer. According to the Savary-Miller classification [Savary, M. and Miller, G., L'oesophage. Manuel et atlas d'endoscopie. Solieure: Gassman (1977)], the esophagitis was grade 4 and regressed to a grade 1 after nine months of treatment using this formulation [Pereira, R. S., J. Pineal Res., 40: 355-356 (2006); Pereira, R. S., J. Pineal Res., 41: 195-200 (2006)].

The regression of GERD symptoms and the relief of the pain may be related to the following: experiments indicate that melatonin has an inhibitory action on gastric acid secretion, and influences ulcer healing, which involves hyperemia at the ulcer margin [Takeuchi, K., et al., Gastroenterology, 106: 1524-1532 (1994)]. Ulcer healing and the gastroprotective effects of melatonin are specifically mediated by the interaction of this indole with melatonin MT2 receptors [Jaworek, J., et al., J. Pineal Res., 38: 73-83 (2005)]. Bubenik and colleagues (1998) demonstrated that a 4-week administration of melatonin in the diet significantly reduced the incidence of spontaneous gastric ulcers in young pigs. The ulcers in this case may have been due to a local deficiency of the melatonin synthesis [Bubenik, G. A., et al., J. Pineal Res., 24: 62-66 (1998)].

Transient lower esophageal sphincter relaxation (TLESR) is a major mechanism of reflux in patients with gastroesophageal reflux disease (GERD). Several agents have been shown to reduce the rate of TLESR, including morphine, somatostatin, nitric oxide synthase inhibitors, among others [Holloway R. H., Am. J. Med., 111: 178S-185S (2001)]. Melatonin inhibits nitric oxide biosynthesis [Jaworek, J., et al., J. Pineal Res., 38: 73-83 (2005)] which may explain the regression of GERD symptoms.

The second agent in the formulation is vitamin B12 (cobalamine), which, in high doses, can alleviate acute pain. Its analgesic effect is attributed to an increased availability and/or effectiveness of noradrenaline and serotonin acting as inhibitory transmitters in the nociceptive system [Jurna, I., Schmerz, 12: 136-141 (1998)]. The third ingredient is folic acid. Epidemiological studies have indicated that this vitamin protects against gastroenterological cancers [Fang, J. Y. and Xiao, S. D., J. Gastroenterol., 38: 821-9 (2003)]. Probably, these agents and other components of the formula (betaine and methionine) induce the synthesis of S-adenosyl-L-methionine (SAMe) [Fetrow, C. W. and Avila, J. R., Ann. Pharmacol., 35: 1414-1425 (2001)], a methyl donor, which has anti-inflammatory as well as analgesic activity without damaging the gastrointestinal mucosa of experimental animals [Gualano, M. et al., Pharmacol. Res. Commun., 15: 683-96 (1983)]. Economically, these biochemicals are less expensive than SAMe and the capsules could be accessible to poorer populations.

1) A dietary supplement for healing or regressing gastroesophageal reflux disease, gastritis and ulcers, by administering to a patient in need of such treatment, containing biochemicals (a mixture of melatonin, vitamins and aminoacids) found in nature and available commercially, which official names given by International Union of Pure and Applied Chemistry (IUPAC) are:
3-Hydroxy-2-methyl-5-([phosphonooxy]methyl)-4-pyridinecarboxaldehyde (Vitamin B6);
L-α-amino-3-indolepropionic acid or 3 β-indolylalanine (Tryptophan);
(Carboxymethyl)trimethylammonium (Betaine);
Pteroyl-L-glutamic acid (Folic Acid or Vitamin M);
α(5,6-dymethylbenzimidazolyl) cyanocobamide (Vitamin B12);
N-Acetyl-5-methoxytryptamine (Melatonin);
L-2-Amino-4-(methylthio)butanoic acid (Methionine).

2) The dietary supplement for healing or regressing symptoms of gastroesophageal reflux disease, gastritis and ulcers, as claimed in claim 1 which is industrialized by the process comprising the following: the biochemicals of the formula described in claim 1 are weighed and encapsulated in gelatin capsule using a semi-automatic, automatic or manual encapsulator. This formulation can be used in other industrial pharmaceutical preparations: pills, tablets, syrups, solutions, injectables, or other similars that can be invented in the future.

3) The dietary supplement of claim 1 which the current formula the action mechanism of ulcers healing based on the fact that melatonin has an inhibitory action on gastric acid secretion, and influences ulcer healing which involves hyperemia at the ulcer margin and it has the action mechanism of gastroesophageal reflux disease based on the fact that melatonin inhibits nitric oxide biosynthesis which may explain the regression of gastroesophageal reflux disease symptoms.

4) The dietary supplement of claim 1 can also be used for sleep disorders such as insomnia, due to sleep-inducing activity of melatonin.

5) The dietary supplement of claim 1 which the current formula has the action mechanism based on the fact that Vitamin B6, B12 and tryptophan, in high doses, can alleviate acute pain. Its analgesic effect is attributed to an increased availability and/or effectiveness of noradrenaline and serotonin acting as inhibitory transmitters in the nociceptive system.

6) The dietary supplement of claim 1 contains folic acid which protects against gastroenterological cancer.

7) The dietary supplement of claim 1 which the current formula has capacity to improve health conditions of HIV positive patients.

8) The dietary supplement of claim 1 in which the concentration of biochemicals of the current formula should be determined via results from analysis of nail topography using atomic force microscopy and dosage of 5-hydroxyindoleacetic acid in the blood of the patients.

9) The dietary supplement of claim 1 in which the current formula has to have a period from 01 to 200 days to start the healing process.
